(19) United States
(12) Patent Application Publication
(10) Pub. No.: US 2007/0166184 A1
(43) Pub. Date: Jul. 19, 2007

(54) Methods for cleaning and sterilizing devices

(75) Inventors: Mohsen Panah, Warsaw, IN (US); Brad L. Rauguth, North Webster, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: Zimmer Technology, Inc.

(21) Appl. No.: 11/331,686

(22) Filed: Jan. 13, 2006

(51) Int. Cl. A61L 2/04

(52) U.S. Cl. 422/1

(57) ABSTRACT

Methods for cleaning and sterilizing devices, such as medical implants and prosthesis, surgical instruments and laboratory equipment. A method of the present invention includes the steps of placing the devices in a chamber of a furnace, evacuating the chamber of the furnace, heating the chamber of the furnace to a depyrogenation temperature, maintaining the chamber at the depyrogenation temperature for a depyrogenation time period, and introducing a flow of a sweep gas through the chamber of the furnace. The depyrogenation temperature may be between 250° C. and 1000° C. and the depyrogenation time period may be between about 45 minutes and 2.0 hours. The sweep gas may comprise any inert gas such as Nitrogen, Argon, or any combination thereof.

### BACKGROUND

The present invention relates to methods for cleaning and sterilizing devices such as medical implants and prostheses, surgical instruments and laboratory equipment.

Several processes have been developed for upgrading (removing bacteria) and cleaning devices, such as medical instruments, prostheses, and laboratory equipment. One method is a simple dry heat upgrade and cleaning method, which involves exposing the devices to dry, high heat for a period of time to kill the bacteria. Although this method may effectively kill the bacteria, the remains of the bacteria may not be removed from the devices.

Another commonly used cleaning method uses nitric acid to clean the devices. In this method, the devices are placed on racks and then immersed in nitric acid. The devices then undergo a series of rinses with reverse osmosis (“RO”) water and are blown dry with compressed air. The devices are further dried in an oven and, then, packaged in sterile containers. Although relatively effective, when used to clean porous devices, the nitric acid may not permeate all of the pores of the device and, thus, may not reach the bacteria that may reside within some pores. When the nitric acid does permeate pores of the device, the water rinses may not remove all of the nitric acid, leaving an undesirable residue of nitric acid in some of the pores. In this case, the nitric acid may seep out from the pores during the heat drying and stain the surface of the device. In addition, this nitric acid cleaning process uses a significant amount of nitric acid, which may present issues regarding the cost and proper disposal of the nitric acid. Further, a significant amount of water is needed for the rinses in this nitric acid process, therefore, requiring water quality testing. Accordingly, a need remains for a more efficient and effective means for upgrading and cleaning devices.

### SUMMARY

The present invention provides methods for cleaning and sterilizing devices such as medical implants and prosthesis, surgical instruments and laboratory equipment. In one embodiment, a method of the present invention includes the steps of placing the devices in a chamber of a furnace, evacuating the chamber of the furnace, heating the chamber of the furnace to a depyrogenation temperature, maintaining the chamber at the depyrogenation temperature for a depyrogenation time period, and introducing a flow of a sweep gas through the chamber of the furnace. In one aspect of this embodiment, the depyrogenation temperature is between 250° C. and 1000° C. and the depyrogenation time period is between about 45 minutes and 2.0 hours. The sweep gas may comprise Nitrogen, Argon, or any other inert gas, or combination thereof and the flow of the sweep gas may be introduced at a flow rate of between 15 gal/min (946 mL/sec) and 25 gal/min (1577 mL/sec).

In another embodiment, the present invention provides a method for destroying and removing pryogens from devices including the steps of placing the devices in a chamber of a furnace, destroying the pryogens by heating the chamber to a depyrogenation temperature and maintaining the chamber at the depyrogenation temperature for a depyrogenation time period, and removing the destroyed pyrogens from the chamber by flowing a sweep gas through the chamber.

In yet another embodiment, the present invention provides a method for sterilizing devices including the steps of placing the devices in a chamber of a furnace, evacuating the chamber of the furnace, heating the chamber of the furnace to a depyrogenation temperature, maintaining the chamber at the depyrogenation temperature for a depyrogenation time period, and introducing a flow of an inert gas through the chamber of the furnace

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a flow chart illustrating the steps of a method according to one embodiment of the present invention.

Corresponding reference characters indicate corresponding parts throughout the several views. Although the drawings represent embodiments of the present invention, the drawings are not necessarily to scale and certain features may be exaggerated in order to better illustrate and explain the present invention. Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

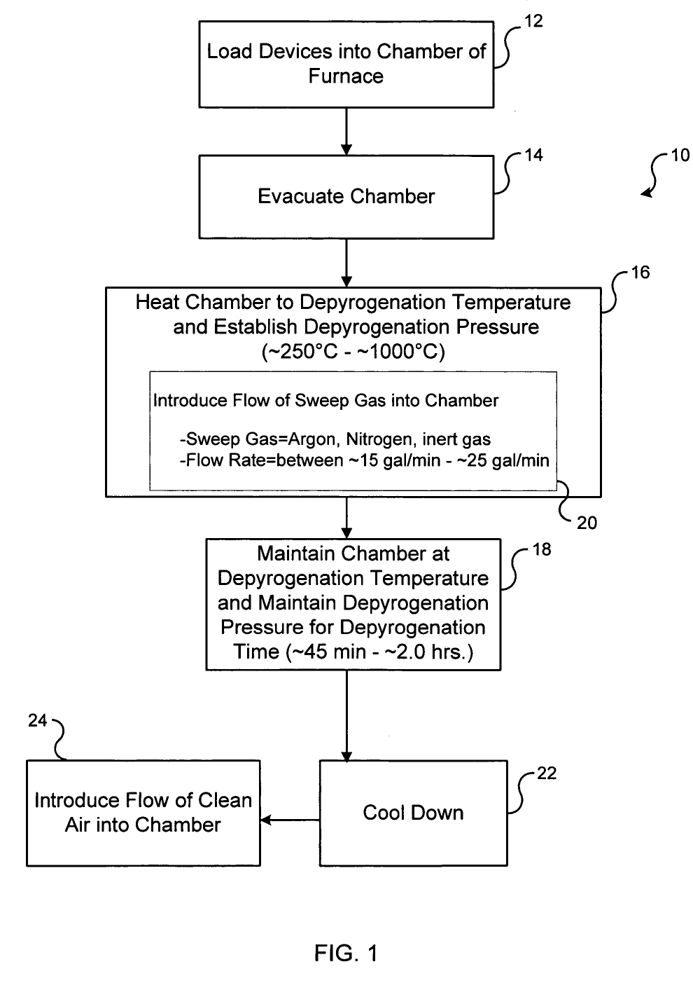

Referring to FIG. 1, a method of cleaning devices according to the present invention will now be described. Method 10, illustrated schematically in FIG. 1, generally includes the steps of loading devices into the chamber of a furnace (12), evacuating the chamber (14), heating the chamber to a depyrogenation temperature and establishing a depyrogenation pressure (16), introducing a flow of a sweep gas into the chamber (20), maintaining the chamber at the depyrogenation temperature for a depyrogenation time (18), and cooling down and unloading the devices from the chamber (22). Method 10 may also include a back-fill step during which a flow of clean air is introduced into the chamber (24) to displace the sweep gas. Each of these steps will now be described in further detail below.

The step of loading the devices into the chamber of a furnace (12) simply involves opening the loading/front door of a suitable furnace, placing the devices to be cleaned into the chamber of the furnace and closing the loading door. The devices to be cleaned may include any device, item, tool or object that needs to be cleaned or sterilized. For instance, the devices may be medical devices, such as implants and surgical tools. The devices may include laboratory equipment such as flasks, plates or other equipment. The devices may be formed of materials such as metal, glass, ceramic or any other material capable of enduring the heat of the depyrogenation temperature. A suitable furnace may be any furnace capable of heating the chamber to, and maintaining the chamber at, a depyrogenation temperature for a depyrogenation time, as further described below. The suitable furnace should also be equipped to receive a flow of a sweep gas as set forth in further detail below. It is further beneficial for the furnace to be capable of evacuating the chamber and maintaining the pressure within the chamber at the depyrogenation temperature discussed in further detail below.

For example, in one embodiment the suitable furnace includes a double-walled, water-cooled vacuum chamber having a front/loading door at one end and a back/unloading door at the other end. The chamber is sealed when both front and back doors are closed and meets a leak rate standard of 5 microns or less per hour. The furnace extends through a wall separating a loading room from a clean (sterile) room such that the back door of the chamber opens into the clean room, where the cleaned devices may be removed from the chamber and either used or packaged in a clean (sterile) environment.

The furnace of this exemplary embodiment also includes a heating system adapted to evenly heat the chamber to the depyrogenation temperature. The heating system may include a radiant heating apparatus and/or a convection apparatus. The furnace also includes a vacuum pump system for evacuating the chamber and control valves for controlling, maintaining, and relieving the pressure within the chamber. The furnace is equipped with a gas delivery system and flow control valves for delivering and controlling the flow of sweep gas and clean air. Finally, the furnace of this exemplary embodiment is equipped with a control system, including an interactive software application and PLC controller that cooperate to provide, store and automatically run cleaning programs. Each cleaning program includes settings such as depyrogenation temperature, depyrogenation time, pressure, sweep gas flow, clean air back fill, and quench time. The control system automatically varies and controls the environment within the chamber according to the program selected.

Referring to FIG. 1, once the devices to be cleaned are loaded into the chamber of a suitable furnace, the front loading door is closed, thereby sealing the chamber. Then, the chamber is evacuated (14) to a minimum of about 10×10⁻⁶Torr.

Next, the temperature within the chamber is heated (ramped up) to a depyrogenation temperature. At this point, a depyrogenation pressure may also be established within the chamber (16). The depyrogenation temperature may be any temperature effective to destroy or kill bacterial endotoxins and/or pryogens. The depyrogenation temperature used may vary depending on the composition of the devices being cleaned and the time for which the devices are exposed to the depyrogenation temperature (i.e., the depyrogenation time). For example, as disclosed in Parenteral Drug Association, Inc.'s, Technical Report No. 7, “Depyrogenation,” 1985, hereby incorporated by reference, indicates that temperatures as low as 140° C. have been used for sterilization of certain substances. Of further example, Inspector's Technical Guide No. 40, “ITG Subject: Bacterial Endotoxins/Pyrogins,” U.S. Food & Drug Administration, Department of Health, Education and Welfare Public Health Service, Mar. 20, 1985, hereby incorporated by reference, temperatures as low as 180° C. may serve as an effective depyrogenation temperature for certain materials, such as glass. Temperatures in the range of 250° C. to 1000° C. are believed to be particularly effective depyrogenation temperatures in the methods of the present invention. More particularly, temperatures in the range of 250° C. to 593° C. have been found to be particularly effective when using the methods of the present invention to clean prosthetic devices made of metal, and a temperature of about 400° C. has proven to be especially effective and practical as a depyrogenation temperature for this purpose. However, it should be understood that any temperature capable of destroying or killing bacterial endotoxins in the method of this invention can serve as the depyrogenation temperature.

Similarly, the pressure useful as a depyrogenation pressure in the methods of the present invention may vary depending on the composition of the devices to be cleaned, the depyrogenation temperature, and the structure of the fturnace chamber. For example, pressures in the range of from about 0.1 Torr (0.0001333 Bar) to about 9,000 Torr (12 Bar) may serve as the depyrogentation pressure. This pressure range is merely exemplary, as noted above the pressure useful as a depyrogenation pressure may vary depending on several factors.

Once the chamber is heated to the depyrogenation temperature (16), the chamber is maintained at the depyrogenation temperature for a depyrogenation time (18), thereby killing and/or destroying bacterial endotoxins on the devices. It may also be beneficial to maintain the chamber at the depyrogenation pressure for the depyrogenation time. As noted above, the depyrogenation time may vary depending on the composition of the devices being cleaned and the depyrogenation temperature. For instance, the depyrogenation time may range from 30 minutes to over 2 hours. A depyrogenation time of between about 1 hour to 2 hours has been found to be particularly effective when cleaning metal prosthetic devices at depyrogenation temperatures between 250° C. and 593° C.

As mentioned above, the cleaning methods of the present invention also include the step of introducing a flow of a sweep gas into the chamber of the furnace. In this step, the sweep gas removes toxins vaporized during the depyrogenation time from the chamber. This step is particularly useful when cleaning porous devices, because the sweep gas can enter the pores of the porous devices and flush out any toxins present therein without leaving undesirable residue as is commonly left behind in conventional rinse/wash cleaning methods. As shown in FIG. 1, the step of introducing the flow of the sweep gas (20) may begin simultaneously with the start of heating the chamber (16). Alternatively, the sweep gas may be introduced at any time during the ramping up of the temperature to the depyrogenation temperature. Although method 10 shows the sweep gas being introduced (20) during the step of heating the chamber (16), alternatively, the sweep gas may be introduced during the step of maintaining the chamber at the depyrogenation temperature (18). The sweep gas may comprise any gas capable of removing the vaporized toxins from the chamber of the furnace without adversely affecting the composition or quality of the devices being cleaned. Gases suitable for use as a sweep gas may comprise inert gases such as Nitrogen, Argon, or any other inert gas, or any combination thereof. The sweep gas may be introduced at any flow rate effective to remove the vaporized toxins from the chamber of the furnace. For instance, flow rates of about 2 gal/min (152 mL/sec) to 50 gal/min (3788 mL/sec) may be used. More particularly, flow rates of between about 15 gal/min to 25 gal/min (1577 mL/sec) have been found to be effective, and a flow rate of about 20 gal/min (1262 mL/sec) has proven to be particularly effective. The flow of the sweep gas is maintained for a time sufficient to remove the vaporized toxins from the chamber. In a particular embodiment, the flow of sweep gas is maintained until the end of the depyrogenation time.

At the end of the depyrogenation time, the flow of the sweep gas is turned off and the step of cooling the chamber of the furnace (22) is performed. The chamber may be cooled by any means. For instance, to facilitate and expedite this step (22), the furnace may be equipped with a quenching system, including a heat exchanger and gas cooling fan, that blows and recirculates cooled gas through the chamber until the chamber is cooled. Once the chamber is cooled, the back unload door is opened and the devices may be unloaded into the clean room for sterile packaging or use.

As illustrated in FIG. 1, method 10 may also include optional back-fill step (24). In back-fill step (24) a flow of clean air is introduced into the chamber after the flow of sweep gas is turned off and just prior to opening the chamber to the clean room. The clean air flow displaces any sweep gas in the chamber and/or in the pores of any porous devices.

The methods of the present invention may be used in many applications. For example, the methods of the present invention may be employed to clean and sterilize medical implants and surgical instrumentation in preparation for sterile packaging. The methods of the present invention may also be used to clean and sterilize surgical instruments or laboratory equipment in preparation for use.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for cleaning devices, the method comprising the steps of:
placing the devices in a chamber of a furnace;
evacuating the chamber of the furnace;
heating the chamber of the furnace to a depyrogenation temperature;
maintaining the chamber at the depyrogenation temperature for a depyrogenation time period; and
introducing a flow of a sweep gas through the chamber of the furnace.

2. The method of claim 1 wherein the sweep gas comprises Argon.

3. The method of claim 1 wherein the sweep gas comprises Nitrogen.

4. The method of claim 1 wherein the flow of the sweep gas comprises a flow rate of between 15 gal/min (946 mL/sec) and 25 gal/min (1577 mL/sec).

5. The method of claim 4 wherein the flow rate is about 20 gal/min (1262 mL/sec).

6. The method of claim 1 wherein said step of introducing the flow of the sweep gas is performed during said step of maintaining the chamber at the depyrogenation temperature.

7. The method of claim 1 wherein the depyrogenation temperature is between 250° C. and 1000° C.

8. The method of claim 7 wherein the depyrogenation temperature is about 400° C.

9. The method of claim 1 wherein the depyrogenation time period is between about 30 minutes and 2.0 hours.

10. The method of claim 9 wherein the depyrogenation time period is about 2 hours.

11. The method of claim 1 further comprising the step of introducing a flow of clean air through the chamber following the step of introducing the flow of the sweep gas.

12. A method for destroying and removing pryogens from devices, the method comprising the steps of:
placing the devices in a chamber of a furnace;
destroying the pryogens by heating the chamber to a depyrogenation temperature and maintaining the chamber at the depyrogenation temperature for a depyrogenation time period; and
removing the destroyed pyrogens from the chamber by flowing a sweep gas through the chamber.

13. The method of claim 12 wherein the depyrogenation temperature is at least 250° C.

14. The method of claim 13 wherein said step of removing the destroyed pyrogens includes flowing the sweep gas through the chamber at a flow rate of between 15 gal/min (946 mL/sec) and 25 gal/min (1577 mL/sec).

15. The method of claim 12 further comprising the step of evacuating the chamber of the furnace.

16. The method of claim 12 further comprising the step of removing the sweep gas from the chamber by flowing clean air through the chamber.

17. The method of claim 12 wherein the sweep gas comprises Argon.

18. A method for sterilizing devices, the method comprising the steps of:
placing the devices in a chamber of a furnace;
evacuating the chamber of the furnace;
heating the chamber of the furnace to a depyrogenation temperature;
maintaining the chamber at the depyrogenation temperature for a depyrogenation time period; and
introducing a flow of a sweep gas through the chamber of the furnace, wherein the sweep gas comprises an inert gas.

19. The method of claim 18 wherein the depyrogenation temperature is between 250° C. and 1000° C.

20. The method of claim 18 wherein the sweep gas comprises Argon.
